(19) United States
(12) Patent Application Publication
(10) Pub. No.: US 2025/0120819 A1
(43) Pub. Date: Apr. 17, 2025

(54) FIBROCARTILAGE REPLACEMENT DEVICE

(71) Applicant: NovoPedics, Inc., Princeton, NJ (US)

(72) Inventors: Cori Lichter, Keyport, NJ (US); Kevin Weadock, Hillsborough, NJ (US)

(73) Assignee: NovoPedics, Inc., Princeton, NJ (US)

(21) Appl. No.: 18/722,670

(22) PCT Filed: Dec. 21, 2022
(86) PCT No.: PCT/US2022/053667

§ 371 Date: Jun. 21, 2024

Related U.S. Application Data
(60) Provisional application No. 63/293,477, filed on Dec. 23, 2021.

(51) Int. Cl. A61F 2/38; A61F 2/30

(52) U.S. Cl. CPC A61F 2/3872; A61F 2002/30062; A61F 2002/30766

(57) ABSTRACT

A scaffold for fibrocartilage replacement is characterized by a polymeric fiber lattice containing circumferential and/or radially oriented regions embedded, placed, or otherwise located in a bioresorbable matrix or adjacent to the surface of a bioresorbable matrix, where the matrix is comprised of biopolymers, such as one or more of collagen and hyaluronic acid. Circumferential or radially oriented fiber regions can intersect at node channels within or through the scaffold.

### CROSS REFERENCE TO RELATED APPLICATIONS

This application claims benefit to U.S. Provisional Application No. 63/293,477 entitled “Fibrocartilage Replacement Device” filed Dec. 23, 2021. The full disclosure of this application is incorporated herein by reference.

### STATEMENT REGARDING FEDERALLY SPONSORED RESEARCH

The present disclosure was made, in part with United States Government support under PID #827392 SUB #1322, awarded by the U.S. Army Medical Research Acquisition Activity (USAMRAA). The United States government may have certain rights in the present disclosure.

### FIELD

The present disclosure relates to methods and devices for replacement of severely damaged fibrocartilage tissue, and in particular, to replacement the lateral or medial meniscus of the knee.

### BACKGROUND

The menisci are two C-shaped discs of fibrocartilage found between the condyles of the femur and the tibial plateau which play a critical role in the load transmission, load distribution, shock absorption, joint stability, and lubrication of the knee. Despite the recognized importance of the tissue, arthroscopic removal of a torn meniscus is one of the most common orthopedic procedures performed in the United States, with almost one million meniscal surgeries performed annually. The public health burden and long-term effects of meniscal injuries and defects are substantial, particularly in physically active populations, because of the increased risk for early-onset osteoarthritis and long-term disability, and the health care costs associated with degenerative joint disease. Because the meniscus has limited healing potential, the clinical outcomes of subtotal meniscectomies are generally poor. At this time, a reliable surgical procedure to replace significant loss of meniscal tissue does not exist. There is no autologous procedure to replace the meniscus and the results of allograft replacement are unreliable.

Another approach is that of tissue engineering. Current approaches include synthetic polymer scaffolds and collagen meniscus implants. With synthetic polymer scaffolds, polyurethane sponges are used to replace the meniscus. This approach has led to inconsistent results. Fibrocartilage growth is seen in some studies using this technology while in others fibrous tissue did not remodel into fibrocartilage. The underlying cartilage was protected in some studies but not protected in others. Another type of meniscus implant uses a sponge containing collagen, hyaluronic acid and chondroitin sulfate. There is promising preliminary data for this implant, but it is not widely accepted by the orthopedic community because of issues with cytotoxic byproducts of cross-linking and scaffold shrinkage. Both of these approaches generate an amorphous structure, the mechanical properties of which may not be appropriate for a device designed to replace the meniscus. Thus, while scaffold technology holds promise, no methods have met with the clinical success necessary for acceptance by the orthopedic community.

U.S. Pat. No. 9,078,756 and WO 2021/092391 both describe a tissue engineered scaffold with the necessary mechanical properties and biocompatibility for treatment of significant meniscal damage and are both incorporated by reference herein in their entirety.

U.S. Pat. No. 9,078,756 discloses a composite meniscal scaffold comprised of a collagen-hyaluronic acid matrix reinforced with a single or primary polymeric fiber that is arranged in circumferential and non-circumferential directions within the matrix of the scaffold.

In WO 2021/092391, a fibrocartilage replacement device utilizes a second polymeric fiber to protect the fibers from damage that might occur during implantation of the device in a human knee. The second polymeric fiber is passed through the body of the scaffold at various sites and then wrapped around the device to protect the device from being damaged during implantation.

### SUMMARY

Certain examples of the present disclosure may include a scaffold for fibrocartilage replacement. The scaffold may include a primary polymeric fiber embedded in a bioresorbable matrix. In an example, the scaffold may have a top surface, an anterior end, a posterior end, and a middle arcuate section therebetween defining a curved path between the anterior and posterior ends. In certain examples, the primary polymeric fiber may form a lattice having circumferentially and radially oriented regions within the middle arcuate region of the scaffold. The circumferentially and radially oriented regions may intersect at node channels. In certain examples, at least a portion of a secondary polymeric fiber may pass through at least one node channel in a direction that may be substantially perpendicular to the circumferentially oriented region of the primary polymeric fiber.

Certain examples of the present disclosure may include a scaffold for fibrocartilage replacement. The scaffold may include a primary polymetric fiber embedded in a bioresorbable matrix. In certain examples, the scaffold may a toroidal shape. The primary polymeric fiber may form a lattice that has circumferentially and radially oriented regions within the scaffold. The circumferentially and radially oriented regions may intersect at node channels. In certain examples, at least a portion of a secondary polymeric fiber may pass through at least one node channel in a direction that may be substantially perpendicular to the circumferentially oriented region of the primary polymeric fiber.

Certain examples of the present disclosure may include a system for making a scaffold for fibrocartilage implant. The system may include a lower plate with at least one alignment hole, an upper plate with at least one alignment hole and at least one alignment bolt that passes through the alignment holes of both upper and lower plates. The upper plate may have a recessed well that has a plurality of small diameter holes that pass all the way through the upper plate and one or more large diameter holes that pass all the way through the upper plate. The small diameter holes may be filled with a winding pin and the large diameter holes may be filled with an anchor pin.

Certain examples of the present disclosure may include a method for making a fibrocartilage implant. The method may include aligning an upper plate on a lower plate. The upper plate may include a recessed well having a plurality of small diameter holes and one or more larger diameter holes that pass through the upper plate. One or more winding pins may be inserted in at least a portion of the plurality of small diameter holes. One or more anchor pins may be inserted in at least a portion of the one or more larger diameter holes. The lower plate may be configured to serve as a stop of the one or more winding pins and the one or more anchor pins. A primary polymeric fiber may be wound into one or more patterns around the one or more winding pins and the one or more anchor pins. The lower plate may be removed. A mold plate may be aligned on the upper plate. The mold plate may include a recess having a shape that is substantially similar to the recessed well. A bioresorbable matrix may be formed within the recess. The matrix may surround the primary polymeric fiber to form a scaffold include one or more node channels corresponding to the one or more winding pins and one or more larger diameter channels corresponding to the one or more anchor pins. The scaffold may be removed from the recess. At least a portion of a secondary polymeric fiber may be threaded through at least one node channel of the one or more node channels.

### BRIEF DESCRIPTION OF DRAWINGS

Other objects and advantages of the present disclosure will become apparent to those skilled in the art upon reading the following detailed description and appended claims, in conjunction with the accompanying drawings, in which like reference numerals have been used to designate like elements, and in which:

FIG. 1 is an illustration of a winding platform system, according to an example of the present disclosure;

FIG. 2 is an illustration of a primary polymeric fiber and winding pattern, according to an example of the present disclosure;

FIG. 3 is an illustration of a mold system used in making the fibrocartilage replacement device, according to an example of the present disclosure;

FIG. 4 is an illustration of the fibrocartilage replacement device, according to an example of the present disclosure;

FIG. 5A is an illustration of the fibrocartilage replacement device with a continuous secondary polymeric fiber wound through node channels, according to an example of the present disclosure;

FIG. 5B is an illustration of a meniscal replacement scaffold with a plurality of secondary polymeric fibers wound through node channels, according to an example of the present disclosure;

FIG. 5C is an illustration of the fibrocartilage replacement device with a secondary polymeric fiber wound through node channels, according to an example of the present disclosure;

FIG. 5D is an illustration of the fibrocartilage replacement device with a secondary polymeric fiber wound through node channels, according to an example of the present disclosure;

FIG. 5E is an illustration of the fibrocartilage replacement device with a secondary polymeric fiber wound through node channels, according to an example of the present disclosure; and

FIG. 6 is an illustration of an intervertebral disc replacement scaffold with a continuous secondary polymeric fiber wound through node channels, according to an example of the present disclosure.

The figures are for purposes of illustrating examples, but it is understood that the present disclosure is not limited to the arrangements and instrumentality shown in the drawings. In the figures, identical reference numbers identify at least generally similar elements.

### DETAILED DESCRIPTION

The present disclosure describes a fibrocartilage replacement device that may maintain the original architecture of a primary fiber lattice created during manufacturing. The overall device may have the ability to translate axial loads to circumferential hoop stress during the early stages of healing after implantation, typically until or even after new tissue ingrowth can replace the stabilizing function of the matrix of the device.

Accordingly, the present disclosure includes a scaffold for fibrocartilage replacement. Certain other examples include methods of using the scaffold for fibrocartilage replacement. Certain other examples include the device for the manufacture of such a scaffold. Certain other examples include methods of making such a scaffold.

### A. Fibrocartilage Replacement Scaffold

Certain examples include a fibrocartilage replacement medical device (also referred to herein as a scaffold) that may be suitable or configured to maintain an original architecture of a primary fiber lattice created during manufacturing while also being advantageously designed to translate axial loads to circumferential stress (e.g., hoop stress) during the early stages of healing after implantation and until new tissue ingrowth can replace the stabilizing function of the collagen matrix.

A polymeric fiber lattice may convert axial compressive forces on the scaffold to tensile loads on the circumferential polymeric fibers. The polymeric fiber lattice may utilize a placed fiber that, in certain examples, may traverse through a node channel within the lattice so that the architecture of the fiber lattice may be maintained after implantation.

In an example, the scaffold may be a meniscal replacement device including a primary polymeric fiber embedded in a matrix, the matrix having both interior and exterior surfaces as well as an anterior end, posterior end, and a middle section.

In an example, the matrix may be formed from bioresorbable material. Bioresorbable material may be one or more of proteins, proteoglycans, biocompatible synthetic polymers and combinations thereof. In an example, the matrix may be one or more of collagen and hyaluronic acid. In an example, the matrix may include crosslinked type I collagen and hyaluronic acid. In an example, the hyaluronic acid may have a molecular weight between 500,000 and 1,800,000 daltons. In certain examples, the collagen-hyaluronic acid matrix may be crosslinked with an agent such as aldehydes, carbodiimides, and hexamethylene di-isocyanates. In an example, the carbodiimide crosslinking agent may be 1-Ethyl-3-(3-dimethylaminopropyl) carbodiimide (EDC). In an example, the efficiency of the crosslinking reaction with EDC may be improved by using N-hydroxysuccinimide in the crosslinking reaction. Physical methods of crosslinking such as dehydrothermal treatment or ultraviolet light (254 nm) may also be used to crosslink the matrix.

The replacement device can be of any shape, including round, circular, circular with a central opening, toroidal, star, u-shaped or arcuate, spherical, etc. If the device is of a u-shape or arcuate shape, the middle section may define a curved path between the anterior and posterior ends.

In some examples, a primary polymeric fiber may form a lattice of circumferentially and radially oriented regions within the shaped matrix.

In an example, the primary polymeric fiber may be bioresorbable. Suitable bioresorbable primary polymeric fibers can be made from polymers such as polylactic acid, poly (desaminotyrosyl-tyrosine dodecyl ester dodecanoate), poly(desaminotyrosyl-tyrosine dodecyl dodecanedioate), polyglycolic acid, poly(lactic-co-glycolic) acid, polydioxanone polycaprolactone, polyarylate, poly-4 hydroxybutyrate, polydioxanone, polyhydroxybutyrate, and poly (trimethylene carbonate). In an example, the polymer used to make the primary polymeric fiber may be non-resorbable. Suitable polymers for making the non-resorbable primary polymeric fibers can be polytetrafluoroethylene (e.g., Teflon), nylon, silk, polypropylene, or polyethylene terephthalate.

In some examples, oriented regions of a polymeric fiber (e.g., a primary polymeric fiber) may extend between the anterior and posterior ends along a curved path and exit the anterior and posterior ends of the scaffold to form respective anterior and posterior attachment segments.

Moreover, circumferential and radial regions may intersect at node channels. In some examples, one or more node channels may be formed before, during or after manufacturing. In some examples, where the node channels are formed during manufacturing, such node channels may be formed by one or more pins (e.g., winding pins) which are integral or connected to a scaffold manufacturing device, as detailed further below, and such node channels may remain upon removal of the scaffold from the manufacturing device.

When present, a node channel may extend at least partially through the matrix. In some examples, the node channel may extend completely through the matrix.

In certain example, at least a portion of a secondary polymeric fiber may pass at least partially through a node channel in a direction that may be substantially perpendicular to the oriented region (e.g., a circumferentially oriented region) of at least one primary polymeric fiber. In other examples, the secondary polymeric fiber may pass completely through at least one node channel in a direction that may be substantially perpendicular to at least one oriented region of the primary polymeric fiber. When present, a secondary polymeric fiber may facilitate maintenance of the structure of the lattice formed by the primary polymeric fiber after the device has been implanted.

In certain examples that include a secondary polymeric fiber, the secondary polymeric fiber may have the same or different diameter of the primary polymeric fiber. In an example, the same polymer type may be used for the primary and secondary polymeric fibers. Other examples may include a first polymer for the primary polymeric fiber and a second polymer that may differ from the first polymer for the secondary polymeric fiber.

These polymeric fiber lattices may convert axial compressive forces on the scaffolds to tensile loads on the circumferential polymeric fibers.

### B. Fibrocartilage Replacement Scaffold Manufacturing

Certain examples of the present disclosure may include a system for making a scaffold for fibrocartilage implant. The system may include a lower plate with at least one alignment hole, an upper plate with at least one alignment hold and at least one alignment bolt that passes through the alignment holes of both upper and lower plates. In an example, the upper plate may include a recessed well having a plurality of small diameter holes that may pass all the way through the upper plate and two large diameter holes that may pass all the way through the upper plate; one or more of the small diameter holes may be filled with a winding pin and one or more of the large diameter holes may be filled with an anchor pin.

In certain examples, the system for making a scaffold for fibrocartilage implant may include a lower plate with at least one alignment hole, an upper plate with at least one alignment hole, and at least one alignment bolt that passes through the alignment holes of both upper and lower plates and a mold plate. In an example, the system may further include a computer numerical control (CNC) machine with a computer.

In certain examples the winding pins may be made from one or more of stainless steel, titanium, and nitinol. In certain examples, the anchor pins or alignment bolts may be made from one or more of stainless steer, titanium, or polyether ether ketone polymer. In certain examples, the upper plate, lower plate, or mold plate may be made of metal or polymer.

In certain examples, after inclusion of a secondary polymeric fiber, the scaffold device may optionally be reintroduced to an amount of matrix material to fill in one or more node channels and/or to smooth at least a portion of the surface of the scaffold device. The additional matrix material may be the same or different from the original matrix material of the scaffold device, detailed above.

The additional matrix material may be applied by dipping, spraying, brushing or other suitable techniques. After reintroduction of the matrix material, the scaffold device may undergo further crosslinking, rinsing, and lyophilization. In certain of such examples, the secondary polymeric fiber may be located at, just under or completely under the surface of the implantable scaffold device. In an example, the secondary polymeric fiber may be wound over the collagen—hyaluronic acid matrix and through one or more of the node channels. In an example, the secondary fiber may be placed directly around the circumferential fibers prior to application of the collagen—hyaluronic acid matrix to the device. Since the winding pins may still be in place, the secondary fiber may not be passed through pore channels. Instead, the secondary fiber may be wound through other spaces that exist between the primary polymer winding so that it may envelope one or more circumferential fibers at one or more sites along the arcuate region of the device.

### C. Various Examples are Shown With Reference to the Figures

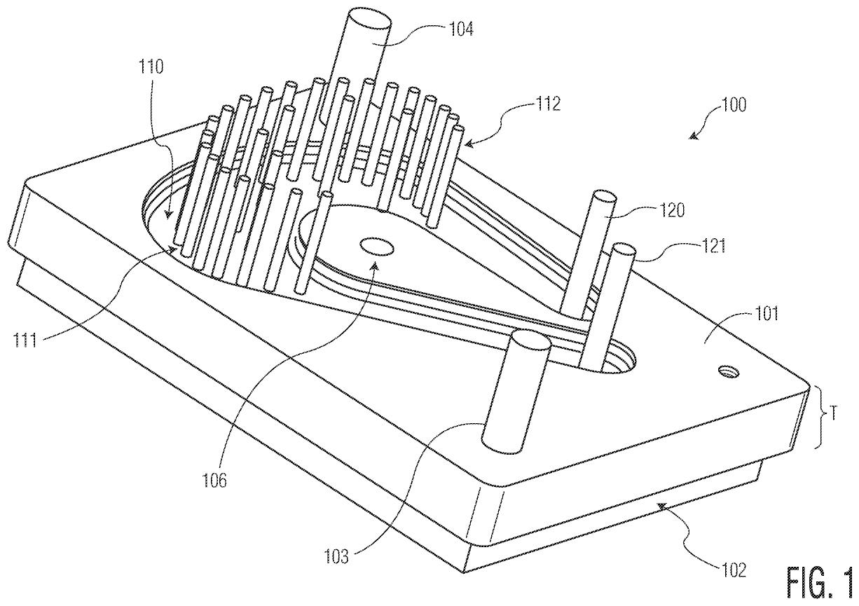

The scaffold for fibrocartilage replacement may be made by use of a winding platform system 100 illustrated in FIG. 1. The winding platform system may be initially set up with a lower plate 102 and an upper plate 101 which may be aligned by one or more alignment bolts 103 and 104 that may pass through alignment holes (not shown) in plates 101 and 102. In an example, only one alignment bolt may be used. In an example, the upper plate or lower plate may be made of a metal or polymer. In an example, the alignment bolts 103 and 104 may have a diameter of approximately 0.10-0.50 inches and a height of approximately 0.20-2.0 inches. In an example, one or both of plates 101 and 102 may be made from 316L stainless steel. In an example, one or both of plates 101 and 102 may be made from a polymer and are formed from 3D printing or injection molding. In an example, one or both of the plates 101 and 102 may have a thickness of approximately 0.20-1.0 inches. In an example, a threaded hole 106 may be drilled in the center of the upper plate 101 and lower plate 102 so that a screw (not shown) may be used to secure the plates together. The alignment bolts 103 and 104 may be mounted on the surface of a CNC machine (not shown) and used to keep the winding platform system 100 registered with the CNC movement.

The upper plate 101 may include a recessed well 110 that may have a plurality of small diameter holes 111 corresponding to node locations for the scaffold to be made. The small diameter holes 111 may pass all the way through the upper plate 101. This design may enable the small diameter holes 111 to be cleaned after the scaffold is made. In an example, the small diameter holes 111 may have a diameter of approximately 0.040-0.070 inches. The small diameter holes 111 may allow winding pins 112 to be inserted therein. In an example, the winding pins 112 may be made of stainless steel. In an example, the winding pins 112 may be made of nitinol. In an example, the winding pins may be made from titanium. In an example, the winding pins 112 may be made of a metal or polymer. In an example, the winding pins 112 may have a length of approximately 0.5-2.0 inches and may have a diameter of approximately 0.035-0.065 inches. Within the recessed well 110 of the upper plate 101, there may be one or more larger diameter holes (not shown) that may allow one or more anchor pins 120 and 121 to be placed. In an example, the anchor pins 112 may have a length of approximately 0.5-2.0 inches and may have a diameter of approximately 0.10-0.50 inches. The lower plate 102 may serve as a stop for the anchor pins 120 and 121 and winding pins 112. In an example, the anchor pins may be made from one or more of stainless steel, titanium, or polyether ether ketone polymer.

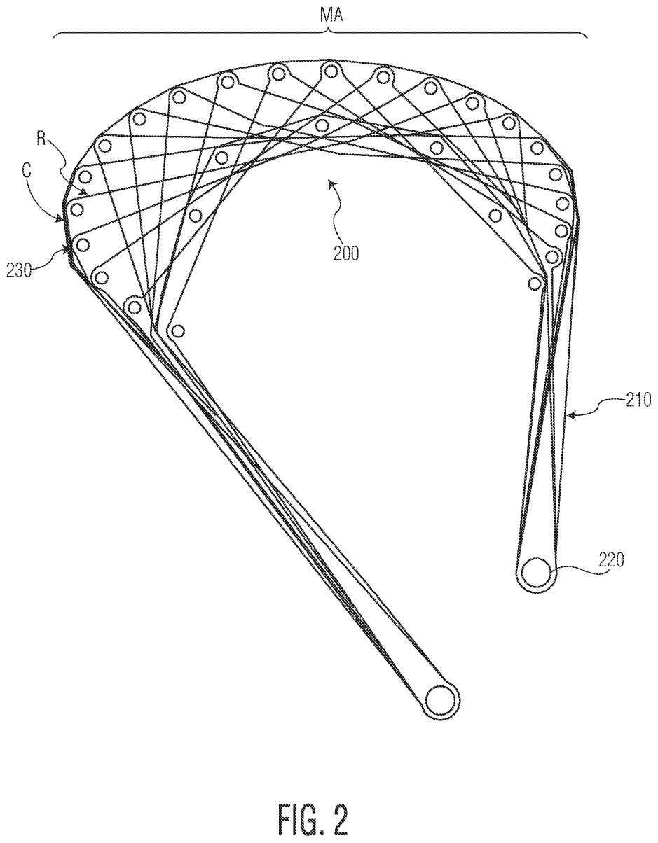

After mounting the winding platform system 100 of FIG. 1 onto the CNC machine (not shown), a first polymeric fiber, also referred to herein as a primary polymeric fiber 210, may be wound into a series of patterns in and around the winding pins by the CNC machine. By way of example, one such pattern 200 is illustrated FIG. 2, which provides a top-down view of the primary polymeric fiber 210, winding pins 230, and anchor pins 220. The primary polymeric fiber 210 may be wound in and around the winding pins 230 and the anchor pins 220. In an example, a code may be used to direct a codable machine (e.g., a CNC machine) to move a primary polymeric fiber 210 along a path which will ultimately produce an architecture that can translate axial loads to circumferential hoop stress. The fiber winding patterns may be prescribed by G-code or other suitable software and may be sent to the CNC machine from a computer. In an example, the primary polymeric fiber 210 may form a lattice of circumferentially oriented regions C and radially oriented regions R within the middle arcuate portion MA of the winding pattern 200, with the circumferentially oriented regions C and radial oriented regions R intersecting at the winding pins 230.

A plurality of different fiber winding patterns of the primary polymeric fiber 210 may be wound in and around the winding pins 230 and anchor pins 220. In an example, up to 80 different fiber winding patterns of the primary polymeric fiber 210 may be wound in and around the winding pins 230 and anchor pins 220. In an example, the effect of the winding pattern may be to translate axial loads applied to the scaffold to circumferential stress withing the primary polymeric fibers of the scaffold. In an example, a single primary polymeric fiber 210 may be used to create all the winding patterns. In another example, additional primary polymeric fibers may be used to make some of the patterns.

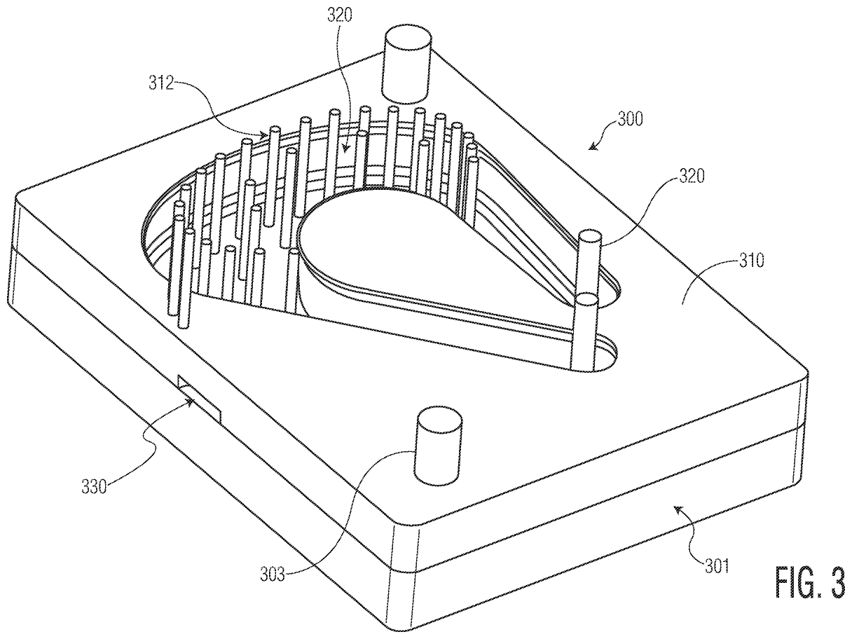

After the fiber winding is completed, the winding platform system 100 illustrated in FIG. 1 may be removed from the CNC machine. The lower plate 102 may be detached from the upper plate 101. A mold plate may be mounted onto the winding platform system to create a mold system. FIG. 3 illustrates the mold system 300 with the mold plate 310 placed on the upper plate 301. Note that the upper plate 301 shown in FIG. 3 may be equivalent to the upper plate 101 shown in FIG. 1. In an example, the mold plate may be made of a metal or polymer.

The alignment bolts 303 and 304 may enable the mold plate 310 to align with the upper plate 301. The mold system 300 may enable a matrix material (not shown) to be added to the mold cavity 320 created by attaching the upper plate 301 to the mold plate 310. The primary polymeric fiber 210 (not shown in FIG. 3) may remain on the winding pins 312 so that the matrix material may be added in and around the primary polymeric fiber 210. A small recess 330 on the side of the mold plate may be used to remove the mold plate 310 from the upper plate 301 at a later time in the process of making the scaffold.

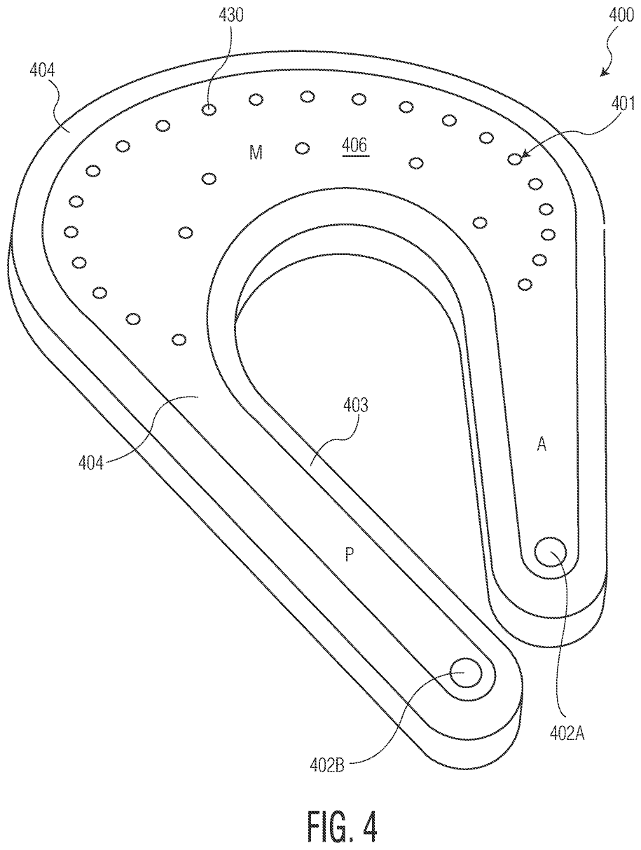

Following the application of the matrix material to the mold, the mold system 300 may be placed in a crosslinking solution so that the matrix material may be crosslinked, exhaustively rinsed in water and buffer solution, and lyophilized. This process of crosslinking, rinsing, and lyophilization process has been described in U.S. Pat. No. 9,078,756, which is incorporated herein by reference. After these chemical processes have been completed, the scaffold may be removed from the mold system by removing the winding pins 230 and anchor pins 220. The resulting scaffold 400, which may include a primary polymeric fiber having both circumferentially and radially aligned regions embedded within a matrix 406, is illustrated in FIG. 4.

The shape and geometry of the scaffold 400 may based on the shape and geometry of a natural human anatomical part in need of replacement (e.g., a meniscus). The scaffold 400 may have an anterior end A, a posterior end P, and a middle arcuate section M therebetween defining a curved path between the anterior end A and posterior end P. In an example, the scaffold 400 may have a beveled interior edge 403 and a beveled exterior edge 404 formed on the top surface 405 of the scaffold 400. The scaffold 400 may have a plurality of node channels 401 formed by the removal of the winding pins 312. One or more other larger diameter channels 402 may be formed by the removal of the anchor pins 320 and may be present at the distal ends of the anterior end A and posterior end P of the scaffold 400. These larger diameter channels 402 may be used by the surgeon to pass sutures or other surgical instruments such as forceps or snares (not shown) through. The sutures or other surgical instruments may then be used to pull the anterior end A and posterior end P of the scaffold 400 through the holes drilled into the tibia.

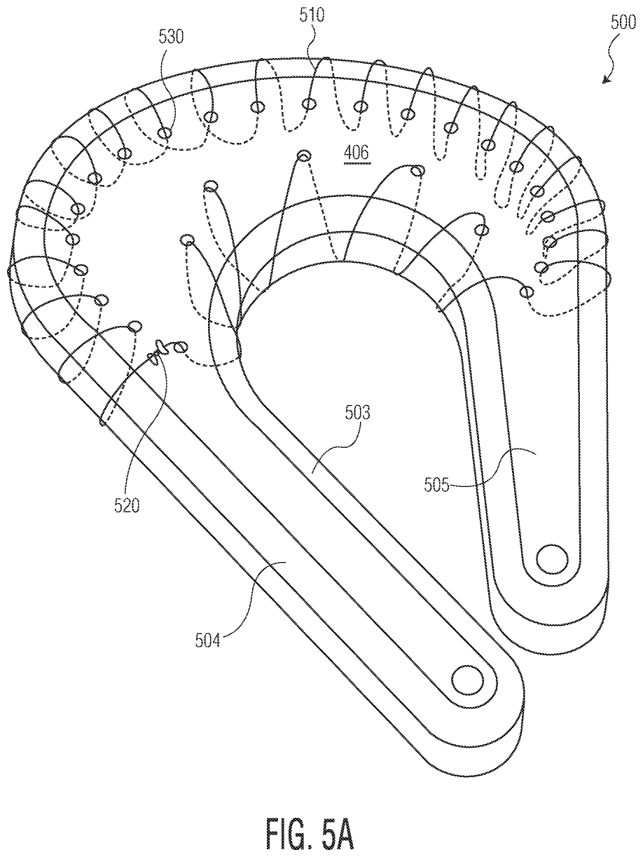

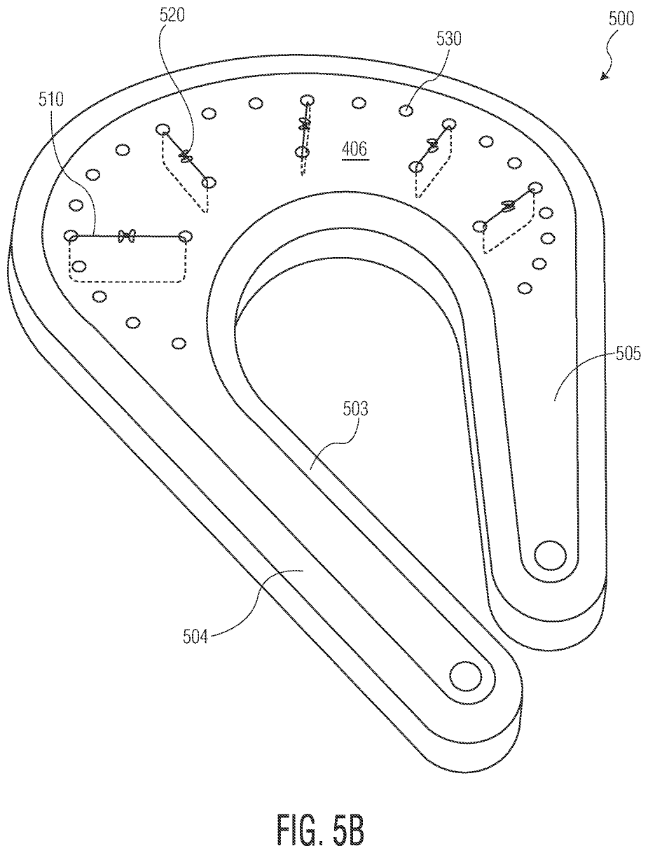

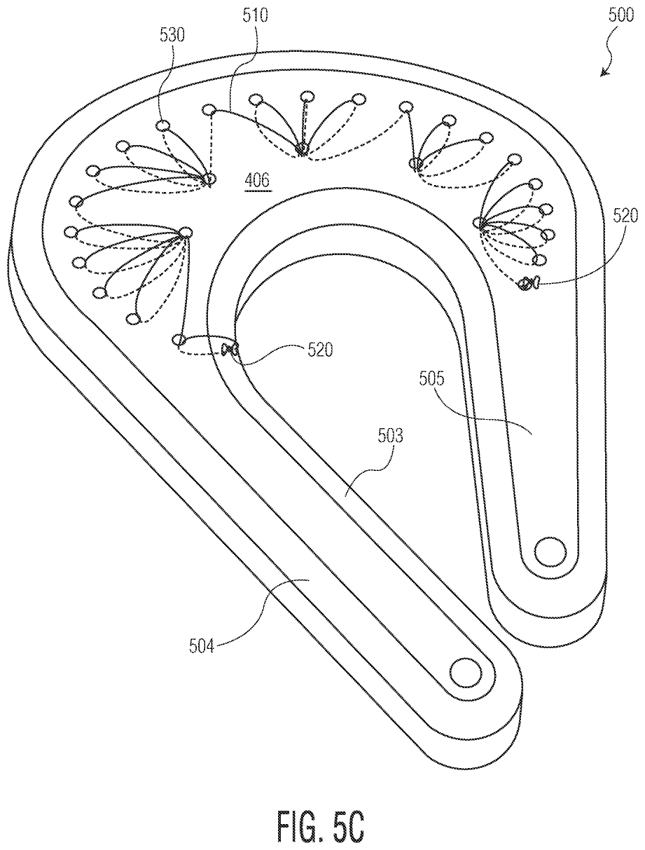

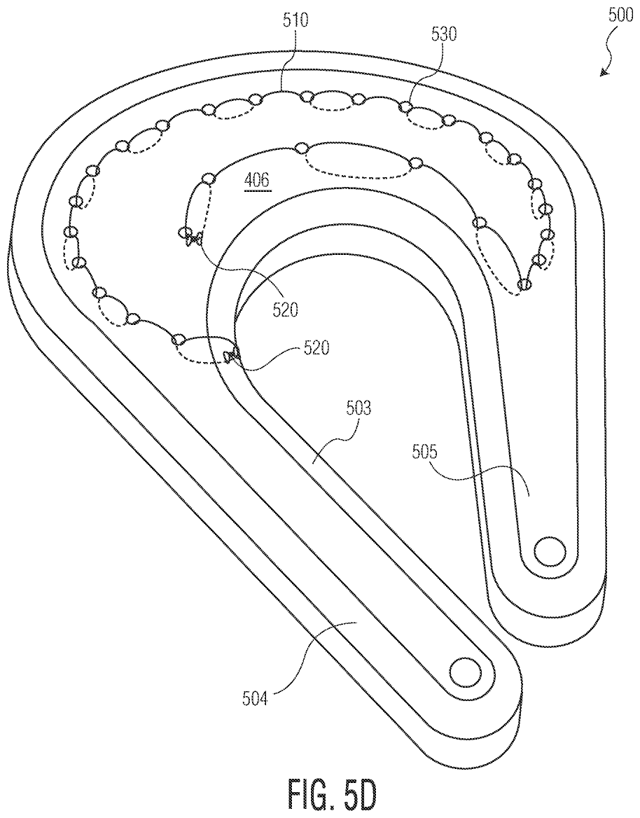

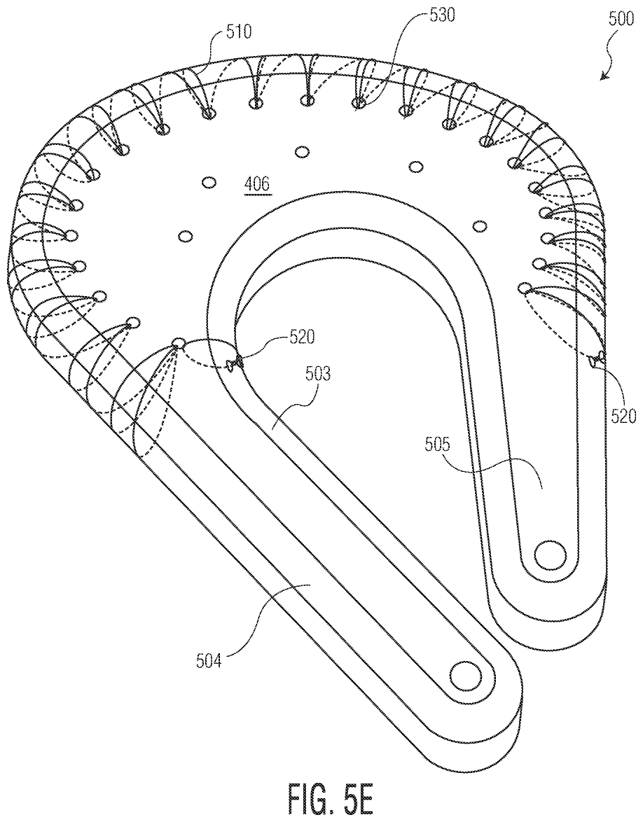

A secondary polymeric fiber 510 may be threaded through one or more of the node channels 530 of the scaffold 400. Different examples of this scaffold 500 are illustrated in FIGS. 5A-5F. It should be noted that the examples in illustrated in FIGS. 5A-5F are not limiting. Any number of secondary polymeric fibers 510 may be passed through any number of the node channels 530 in any pattern.

In an example, the number of times that the secondary polymeric fiber 510 may be passed through a single node channel 530 may be greater than one. In an example, at least a portion of the secondary polymeric fiber 510 may pass through at least one node channel 530 in a direction that may be substantially perpendicular to the circumferentially oriented region of the primary polymeric fiber (shown in FIG. 2). In an example, the ends of the secondary polymeric fiber 510 may be secured together by a knot 520 to ensure that the secondary polymeric fiber 510 remains secured on the scaffold 400. Other means for securing the secondary polymeric fiber 510 may include adhesives, clamps, or buttons. The secondary polymeric fiber 510 may ensure that the structure of the lattice formed by the primary polymeric fiber 210 (shown in FIG. 2), along with its ability to translate axial loads to circumferential hoop stress, may be maintained after the scaffold 500 has been implanted within the patient's knee.

In an example shown in FIG. 5A, the secondary polymeric fiber 510 may be threaded through the node channels 530 of the scaffold 400 and over the beveled interior edge 503 and beveled exterior edge 504 formed on the top surface 405 of the scaffold 400.

In an example shown in FIG. 5B, a plurality of secondary polymeric fibers 510 may be threaded through the node channels 530 only, and may not pass over the beveled interior edge 503 or beveled exterior edge 504 formed on the top surface 405 of the scaffold 400. In an example, one or more ends of the individual secondary polymeric fibers 510 may be ligated or otherwise secured together (e.g., by a knot 520) to ensure that the one or more secondary polymeric fibers 510 remain secured on the scaffold 400.

In an example shown in FIG. 5C, the secondary polymeric fiber 510 may pass through each node channel 530 adjacent to the beveled exterior edge 504 at least once and may pass through each node channel 530 adjacent to the beveled interior edge 503 multiple tines.

In an example shown in FIG. 5D, the secondary polymeric fiber 510 may pass through each node channel 530 adjacent to the beveled exterior edge 504 one by one and then through each node channel 530 adjacent to the beveled interior edge 503 one by one.

In an example shown in FIG. 5E, the secondary polymeric fiber 510 may only pass through each node channel 530 adjacent to the beveled exterior edge 504.

In another example, the scaffold may be an intervertebral disc replacement scaffold where the scaffold may be elliptical or circular shaped with a center opening. The primary polymeric fiber may be wound around pins that form two concentric circles. In an example, the inner circle may surround an elastomeric material that mimics the properties of the native nucleus pulposus. In an example, the scaffold height may be between approximately 0.5 and 1.2 centimeters. After the bioresorbable matrix is added in and around the pins, the scaffold may be removed from the pins. Node channels may remain where the pins once were. In this example, there may be no anterior or posterior ends.

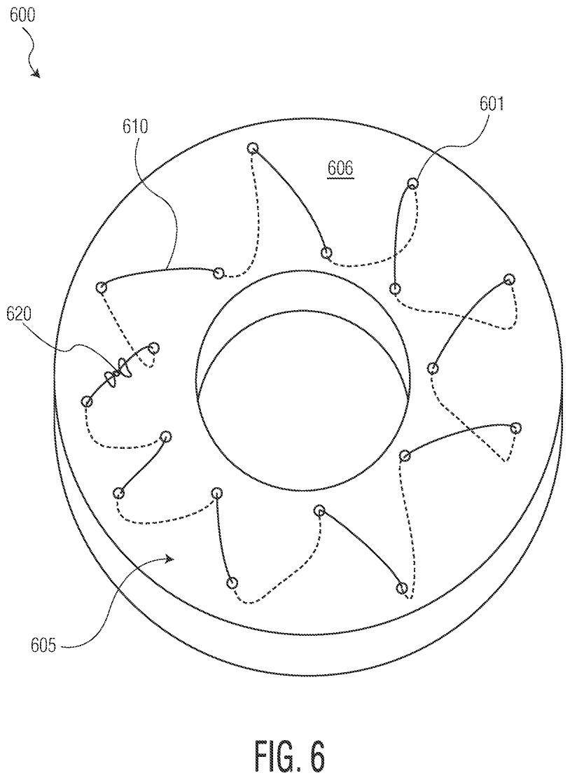

FIG. 6 is an illustration of an intervertebral disc replacement scaffold 600 with a single continuous secondary polymeric fiber 610 wound through node channels 601. The scaffold 600 may be similar to the scaffold 500 described above and formed using similar techniques, but may be a toroidal shape. In an example, one or more ends of the secondary polymeric fiber 610 may be secured together by a knot 620 to ensure that the secondary polymeric fiber 610 remains secured on the matrix 606. Analogous to the examples of FIG. 5A and FIG. 5B, in an example of the intervertebral disc scaffold, the secondary polymeric fiber 610 may have the same diameter of a primary polymeric fiber (not shown) that may be similar to the primary polymeric fiber 210 described above. In an example, the diameter of the secondary polymeric fiber 610 may be between approximately 50 and 200 microns. In an example of the intervertebral scaffold 600, the same polymer may be used for the primary polymeric fiber and secondary polymeric fiber 610.

Post-operative imaging and assessment of the scaffolds described above may be important to both surgeons and patients. In an example, the secondary polymeric fibers described above may be further include, either by incorporation into the polymer or coating of the fiber, a radiopaque material such as iodine, barium, tantalum, bismuth, or gold. In an example of the scaffolds described above, the bioresorbable matrix may contain platelet rich plasma or mammalian cells. In an example of the scaffolds described above, the bioresorbable matrix may contain an antimicrobial agent, antibiotic, or anti-fungal agent. In an example of the scaffolds described above, the bioresorbable matrix may contain bone derivatives or calcium-phosphate compounds.

Although the present disclosure has been described with reference to particular examples, it is to be understood that these examples are merely illustrative of the principles and applications of the present examples. It is therefore to be understood that numerous modifications may be made to the examples and that other arrangements may be devised without departing from the spirit and scope of the present disclosure.

The methods described herein may be performed by a processing device (e.g. smartphone, computer, computing device, etc.). The methods may include one or more operations, functions, or actions as described above. Additional examples of the presently described method and device embodiments are suggested according to the structures and techniques described herein. Other non-limiting examples may be configured to operate separately or may be combined in any permutation or combination with any one or more of the other examples provided above or throughout the present disclosure.

It will be appreciated by those skilled in the art that the present disclosure may be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The terms “including” and “comprising” should be interpreted as meaning “including, but not limited to.” If not already set forth explicitly in the claims, the term “a” should be interpreted as “at least one” and the terms “the, said, etc.” should be interpreted as “the at least one, said at least one, etc.”

The present disclosure is described with reference to methods and devices, which may be implemented by means of analog or digital hardware and computer program instructions stored on a non-transitory computer readable medium. These computer program instructions may be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified.

For the purposes of this disclosure a non-transitory computer readable medium (or computer-readable storage medium/media) stores computer data, which data may include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, cloud storage, magnetic cassettes, magnetic tape, magnetic disk storage or other magnetic storage devices, or any other physical or material medium which may be used to tangibly store the desired information or data or instructions and which may be accessed by a computer or processor.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For the purposes of this disclosure the term “server” should be understood to refer to a service point which provides processing, database, and communication facilities. By way of example, and not limitation, the term “server” may refer to a single, physical processor with associated communications and data storage and database facilities, or it may refer to a networked or clustered complex of processors and associated network and storage devices, as well as operating software and one or more database systems and application software that support the services provided by the server. Cloud servers are examples.

For the purposes of this disclosure, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2^{nd}, 3^{rd}, 4th, or 5^{th} generation (2G, 3G, 4G or 5G) cellular technology, Bluetooth, 802.11b/g/n, or the like. Network access technologies may enable wide area coverage for devices, such as client devices with varying degrees of mobility, for example. In short, a wireless network may include virtually any type of wireless communication mechanism by which signals may be communicated between devices, such as a client device or a computing device, between or within a network, or the like.

It is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

1. A fibrocartilage implant comprising:
a bioresorbable matrix;
a primary polymeric fiber embedded in the bioresorbable matrix to form a scaffold, wherein the primary polymeric fiber forms a lattice comprising a circumferentially oriented region and a radially oriented region within the scaffold, the circumferentially oriented region and the radially oriented region intersecting at one or more node channels of the scaffold; and
a secondary polymeric fiber, wherein at least a portion of the secondary polymeric fiber passes through at least one node channel of the one or more node channels.

2. The fibrocartilage implant of claim 1, wherein one or more of the primary polymeric fiber and the secondary polymeric fiber is a bioresorbable polymer comprising polylactic acid, polyglycolic acid, poly(lactic-co-glycolic) acid, polycaprolactone, polyarylate, polyhydroxybutyrate, polydioxanone, poly(desaminotyrosyl-tyrosine dodecyl dodecanedioate), and poly (trimethylene carbonate).

3. The fibrocartilage implant of claim 1, wherein the secondary polymeric fiber is non-resorbable.

4. The fibrocartilage implant of claim 1, wherein at least a portion of the secondary polymeric fiber contacts a top surface of the scaffold.

5. The fibrocartilage implant of claim 1, wherein the secondary polymeric fiber passes through a node channel of the one or more node channels two or more times.

6. The fibrocartilage implant of claim 1, wherein the secondary polymeric fiber comprises one or more segments.

7. The fibrocartilage implant of claim 1, wherein the secondary polymeric fiber is secured to the scaffold by a knot, adhesive, clamp, or button.

8. The fibrocartilage implant of claim 1, wherein the bioresorbable matrix is formed from a material comprising one or more of proteins, proteoglycans, biocompatible synthetic polymers and combinations thereof.

9. The fibrocartilage implant of claim 1, wherein the secondary polymeric fiber comprises a radiopaque material containing iodine, barium, tantalum, bismuth, or gold.

10. The fibrocartilage implant of claim 1, wherein the scaffold comprises a top surface, an anterior end, a posterior end, and a middle arcuate section therebetween defining a curved path between the anterior end and the posterior end.

11. The fibrocartilage implant of claim 10, wherein the circumferentially and radially oriented regions are within the middle arcuate section of the scaffold.

12. The fibrocartilage implant of claim 1, wherein the scaffold comprises a toroidal shape.

13. The fibrocartilage implant of claim 1, wherein the at least the portion of the secondary polymeric fiber passes through the at least one node channel in a direction that is substantially perpendicular to the circumferentially oriented region of the primary polymeric fiber.

14. A system for making a fibrocartilage implant, the system comprising:
a lower plate;
an upper plate configured to be aligned with the lower plate, the upper plate comprising a recessed well having a plurality of small diameter holes and one or more larger diameter holes that pass through the upper plate;
one or more winding pins configured to be inserted in at least a portion of the plurality of small diameter holes; and
one or more anchor pins configured to be inserted in at least a portion of the one or more larger diameter holes, the lower plate configured to serve as a stop of the one or more winding pins and the one or more anchor pins.

15. The system of claim 14, wherein the upper plate is aligned with the lower plate via an alignment bolt that passes through an alignment hole of the upper plate and an alignment hole of the lower plate.

16. The system of claim 14, further comprising a mold plate configured to be aligned with the upper plate, the mold plate comprising a recess having a shape that is substantially similar to the recessed well.

17. The system of claim 16, wherein the mold plate is aligned with the upper plate via an alignment bolt that passes through an alignment hole of the mold plate and an alignment hole of the upper plate.

18. The system of claim 16, wherein one or more of the lower plate, the upper plate, and the mold plate are made from a material comprising one or more of a metal and a polymer.

19. The system of claim 14, wherein one or more of the one or more winding pins and the one or more anchor pins are made from a material comprising one or more of stainless steel, titanium, and nitinol.

20. The system of claim 14, wherein the recessed well comprises an anterior end, a posterior end, and a middle arcuate section therebetween defining a curved path between the anterior end and the posterior end.

21. The system of claim 14, wherein the recessed well comprises a toroidal shape.

22. A method for making a fibrocartilage implant, the method comprising:
aligning an upper plate on a lower plate, the upper plate comprising a recessed well having a plurality of small diameter holes and one or more larger diameter holes that pass through the upper plate;
inserting one or more winding pins in at least a portion of the plurality of small diameter holes;
inserting one or more anchor pins in at least a portion of the one or more larger diameter holes, the lower plate configured to serve as a stop of the one or more winding pins and the one or more anchor pins;
winding a primary polymeric fiber into one or more patterns around the one or more winding pins and the one or more anchor pins;
removing the lower plate;
aligning a mold plate on the upper plate, the mold plate comprising a recess having a shape that is substantially similar to the recessed well;
forming a bioresorbable matrix within the recess, the matrix surrounding the primary polymeric fiber to form a scaffold comprising one or more node channels corresponding to the one or more winding pins and one or more larger diameter channels corresponding to the one or more anchor pins;
removing the scaffold from the recess; and
threading at least a portion of a secondary polymeric fiber through at least one node channel of the one or more node channels.

23. The method of claim 22, wherein the aligning the upper plate on the lower plate comprises inserting an alignment bolt hat extends through an alignment hole in the upper plate and an alignment hole in the lower plate.

24-39. (canceled)
